(19) United States
(12) Patent Application Publication
(10) Pub. No.: US 2020/0335206 A1
(43) Pub. Date: Oct. 22, 2020

(54) MEDICAL TREATMENT PLAN SUPPORT APPARATUS

(71) Applicant: CANON MEDICAL SYSTEMS CORPORATION, Otawara-shi (JP)

(72) Inventor: Yoshifumi YAMAGATA, Sakura (JP)

(73) Assignee: CANON MEDICAL SYSTEMS CORPORATION, Otawara-shi (JP)

(21) Appl. No.: 16/844,001

(22) Filed: Apr. 9, 2020

(30) Foreign Application Priority Data
Apr. 19, 2019 (JP) 2019-080426

(51) Int. Cl. G16H 40/20; G16H 20/00; G16H 10/60; G16H 15/00; G16H 50/70; G16H 50/20; G06Q 40/08; G06Q 30/02

(52) U.S. Cl. CPC G16H 40/20; G16H 20/00; G16H 10/60; G06Q 30/0283; G16H 50/70; G16H 50/20; G06Q 40/08; G16H 15/00

(57) ABSTRACT

A medical treatment plan support apparatus according to an embodiment includes processing circuitry and a hardware display device. The processing circuitry generates a plurality of medical treatment plans including medical treatment actions provided by a plurality of medical institutions to a patient, on the basis of patient information indicating a state of the patient and medical institution information regarding the medical institutions. The processing circuitry causes the hardware display device to display the medical treatment plans in a comparable manner. The hardware display device displays the medical treatment plans in a comparable manner under control of the processing circuitry.

### CROSS-REFERENCE TO RELATED APPLICATIONS

This application is based upon and claims the benefit of priority from Japanese Patent Application No. 2019-080426, filed on Apr. 19, 2019; the entire contents of which are incorporated herein by reference.

### FIELD

Embodiments described herein relate generally to a medical treatment plan support apparatus.

### BACKGROUND

In prior art, there are known techniques that are capable of searching for information, such as diseases that can be treated in each of a plurality of medical treatment institutions, past medical treatment results of a plurality of medical treatment institutions, and the like. In addition, in recent years, attention is paid to a family doctor system, in which the family doctor of a patient generates a medical treatment plan for the patient in cooperation with other medical institutions, as well as with the medical institution for which the family doctor works.

However, when a plurality of choices for a treatment method exist, or when available medical treatment actions are different among medical institutions, it is not easy for the family doctor to generate a plurality of medical treatment plans in consideration of these pieces of information.

### BRIEF DESCRIPTION OF THE DRAWINGS

FIG. 1 is a diagram illustrating an example of relation between a plurality of medical institutions according to a first embodiment;

FIG. 2 is a block diagram illustrating an example of functions of a medical treatment plan support system according to the first embodiment;

FIG. 3 is a diagram illustrating an example of a list screen of a plurality of medical treatment plans according to the first embodiment;

FIG. 4 is a diagram illustrating an example of a list screen displaying medical treatment actions in the medical treatment plans according to the first embodiment;

FIG. 5 is a diagram illustrating an example of adding items to the list screen of the medical treatment plans according to the first embodiment;

FIG. 6 is a flowchart illustrating an example of flow of a medical treatment plan generation process according to the first embodiment;

FIG. 7 is a diagram illustrating an example of a list screen of a plurality of medical treatment plans according to a second embodiment;

FIG. 8 is a diagram illustrating an example of display of the reason why acceptance is impossible according to the second embodiment;

FIG. 9A is a flowchart illustrating an example of flow of a medical treatment plan generation process according to the second embodiment;

FIG. 9B is a continuation of the flowchart illustrated in FIG. 9A;

FIG. 10 is a diagram illustrating an example of insurance information according to a third embodiment;

FIG. 11 is a diagram illustrating an example of a list screen of a plurality of medical treatment plans according to the third embodiment; and

FIG. 12 is a diagram illustrating another example of the list screen of a plurality of medical treatment plans according to the third embodiment.

### DETAILED DESCRIPTION

### First Embodiment

According to one embodiment, a medical treatment plan support apparatus comprises processing circuitry. The processing circuitry generates a plurality of medical treatment plans including medical treatment actions provided by a plurality of medical institutions to a patient, on the basis of patient information indicating a state of the patient and medical institution information regarding the medical institutions. The processing circuitry causes a hardware display device to display the medical treatment plans in a comparable manner.

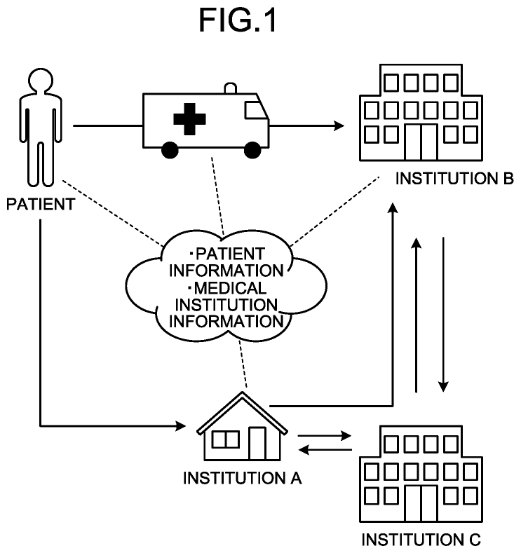

FIG. 1 is a diagram illustrating an example of relation of a plurality of medical institutions according to the present embodiment. As an example, when a plurality of medical institutions exist in an area, there are cases where the medical institutions perform medical treatment for a patient in cooperation with one another.

In the example illustrated in FIG. 1, suppose that an institution A is a clinic for which a family doctor of the patient works. An institution B is a general hospital capable of providing an acute medical care. An institution C is a general hospital providing no acute medical care. Suppose that the institution A, the institution B, and the institution C are located in an area of a certain range. The term “certain range” indicates, for example, the same city, town, or village, but may include a plurality of adjacent cities, towns, and/or villages, as long as the range falls within a distance in which the patient can use the institutions.

In such a case, when the family doctor of the institution A generates a medical treatment plan for the patient, the family doctor may generate a medical treatment plan including medical treatment in the institution B or the institution C, as well as the institution A.

In addition, for example, the patient may go to the institution B to receive an acute medical care. In such a case, the doctor or the like of the institution B may generate a medical treatment plan including medical treatment in the institution A or the institution C for development observation and/or outpatient treatment after the acute phase. As described above, a plurality of medical institutions in the area working in cooperation with one another to provide medical services to patients is called “community-based integrated care”.

The medical treatment plan support apparatus and a medical treatment plan support system according to the present embodiment support the doctor or the like for generation of a medical treatment plan involving a plurality of medical institutions in such cases. For example, the medical treatment plan support apparatus according to the present embodiment shares patient information indicating the state of the patient and medical institution information regarding a plurality of medical institutions, and generates a plurality of medical treatment plans including medical treatment actions provided by a plurality of medical institutions on the basis of these pieces of information.

A medical treatment plan is information including a plan of a medical examination and treatment provided by the medical institution to the patient. In the present embodiment, a medical examination or treatment includes various types of medical services, such as inspections, surgical operations, medication, rehabilitation, and development observation.

In the example illustrated in FIG. 1, the institution A, the institution B, and the institution C are mentioned as an example of a plurality of medical institutions, but the type and the number of medical institutions are not limited thereto. For example, hospitals, home care providers, or the like may be included as the medical institutions.

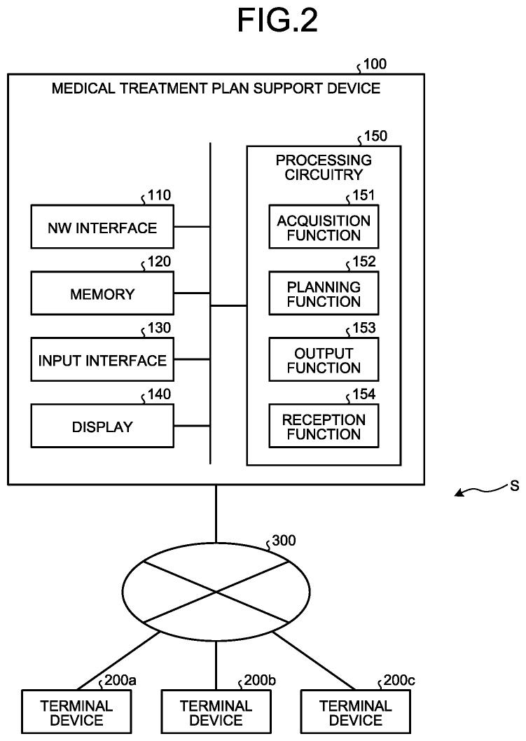

FIG. 2 is a block diagram illustrating an example of functions of a medical treatment plan support system S according to the first embodiment. As illustrated in FIG. 2, the medical treatment plan support system S includes a medical treatment plan support apparatus 100 and a plurality of terminal devices 200a to 200c. The medical treatment plan support apparatus 100 and the terminal devices 200a to 200c are connected via a network 300, such as the Internet.

Each of the terminal devices 200a to 200c is installed in a medical institution. For example, the terminal device 200a is installed in the institution A, the terminal device 200b is installed in the institution B, and the terminal device 200c is installed in the institution C. In the following explanation, when the terminal devices 200a to 200c are not specifically distinguished from each other, each of the terminal devices are simply referred to as a terminal device 200. The number of terminal devices 200 is not limited to the example illustrated in FIG. 2.

The terminal device 200 is, for example, a personal computer (PC) or a mobile terminal or the like. As an example, the terminal device 200 includes: an input interface such as a mouse and a keyboard; a display such as a liquid crystal display and a cathode ray tube (CRT) display; a memory implemented with a semiconductor memory element such as a random access memory (RAM) and a flash memory, a hard disk, and/or an optical disk; and processing circuitry such as a processor.

The terminal device 200 transmits information input by the user, such as the doctor, to the medical treatment plan support apparatus 100 through the network 300. The terminal device 200 also receives a list screen of a plurality of medical treatment plans or various types of information from the medical treatment plan support apparatus 100, and displays the received information on a display. The display of the terminal device 200 serves as an example of the screen in the present embodiment.

The medical treatment plan support apparatus 100 is, for example, a server. The installation place of the medical treatment plan support apparatus 100 is not specifically limited, but may be installed in any of the medical institutions. The medical treatment plan support apparatus 100 may be installed in a cloud environment.

Specifically, the medical treatment plan support apparatus 100 includes a network (NW) interface 110, a memory 120, an input interface 130, a display 140, and processing circuitry 150.

The NW interface 110 is connected with the processing circuitry 150, and controls transmission of various data and communication performed between the medical treatment plan support apparatus 100 and the terminal apparatus 200. The NW interface 110 is implemented with a network card, a network adaptor, and/or a network interface controller (NIC), or the like.

The memory 120 stores in advance various types of information used in the processing circuitry 150. In the present embodiment, the memory 120 stores patient information and medical institution information therein.

The memory 120 is implemented with, for example, a semiconductor memory element such as a RAM and a flash memory, a hard disk, an optical disk, and the like. The memory 120 is also referred to as a memory unit.

The input interface 130 is, for example, a mouse and/or a keyboard. The input interface 130 receives various input operations from an operator, converts the received input operations into electrical signals, and outputs the electrical signals to the processing circuitry 150. Examples of the input interface 130 also include electrical signal processing circuitry receiving an electrical signal corresponding to an input operation from an external input device provided separately from the medical treatment plan support apparatus 100, and outputting the electrical signal to the processing circuitry 150. The display 140 is a liquid crystal display or a CRT display.

The processing circuitry 150 includes an acquisition function 151, a planning function 152, an output function 153, and a reception function 154. The acquisition function 151 serves as an example of the acquisition unit. The planning function 152 serves as an example of the planning unit. The output function 153 serves as an example of the output unit. The reception function 154 serves as an example of the reception unit.

The acquisition function 151 acquires various types of information from the terminal device 200 through the network 300 and the NW interface 110. For example, the acquisition function 151 acquires patient information and medical institution information regarding each of the medical institutions from the terminal device 200. The acquisition function 151 saves the acquired patient information and medical institution information in the memory 120.

The patient information is the information regarding the patient; or more specifically, the information on the patient such as the age, the gender, the previous history, the history of present illness, and the symptom. The acquisition function 151 may acquire only the patient information on the specific patient serving as a target of generation of medical treatment plans, or acquire respective pieces of patient information on a plurality of patients collectively. Because the patient information described above is information generally recorded on the electronic medical chart, for example, the acquisition function 151 may acquire electronic medical chart information stored in the terminal device 200.

The medical institution information is information regarding a plurality of medical institutions. More specifically, the medical institution information is information including the past medical treatment results in each of the medical institutions, information indicating effects of the past medical treatments in each of the medical institutions, the cost pertaining to the medical treatment actions in each of the medical institutions, information on the income received with each of the medical institutions in accordance with the medical treatment actions, and information regarding medical treatment actions that each of the medical institutions can provide.

The past medical treatment results in each of the medical institutions include: the types of illness and injuries treated in each of the medical institutions in the past, the details of the treatment, the number of patients treated for each of the diseases, and the period of treatment for each of the diseases. The details of the treatment include information regarding, for example, the type of the inspection, the type of the surgical operation, the details of the medication, and information on treatment such as radiation treatment.

The information indicating the effects of the past medical treatments in each of the medical institutions is, for example, the number of days of hospitalization, the recurrence rate, and/or the mortality rate. The number of days of hospitalization is, for example, the average number of days of hospitalization of a plurality of patients for each of types of illness and injuries and each of details of treatments. The recurrence rate or the mortality rate is, for example, the recurrence rate of the disease or mortality of the patients for each of types of illness and injuries and each of details of treatments. The information indicating the effects of the medical treatments may be different according to the type of diseases. The information indicating the effects of the medical treatments may be results totaled for each of attributes, such as the age and the gender of the patients. The information indicating the effects of the past medical treatments in each of the medical institutions may be evaluation of the effects based on the average value in each of the medical institutions, regardless of the type of illness and injuries and the details of the medical treatment.

In the present embodiment, the effect or the result of the medical treatment is referred to as “outcome”.

The cost pertaining to the medical treatment actions in each of the medical institutions is, for example, the labor costs, the pharmaceutical product costs, and equipment costs.

The information on the income received with each of the medical institutions in accordance with the medical treatment actions is the medical treatment fee paid by the patient or the insurance system to the medical institution. In the present embodiment, the sum obtained by subtracting the costs described above from the income serves as the balance, but the sum to which accounting items, such as other equipment related costs, are added may serve as the balance.

The medical treatment actions that each of the medical institutions can provide include the types of illness and injuries that can be treated in the future in each of the medical institutions, the details of treatments available for the patient, or the like. Even a medical treatment action with no past medical treatment results is regarded as an available medical treatment action, if they are planned to be provided in the future.

The medical institution information may also include other types of information regarding the evaluation indexes of the medical institutions. For example, the medical institution information may include the number of occurrences of nosocomial infection in each of the medical institutions or information on the degree of satisfaction answered by the patients.

The planning function 152 generates a plurality of medical treatment plans including medical treatment actions provided to the patient by the medical institutions, on the basis of the patient information and the medical institution information regarding the medical institutions.

For example, there are cases where treatments for the disease that the patient has contracted involve surgical treatment by a surgical operation, internal treatment by medication or radiation treatment, or treatment using both surgical treatment and internal treatment in combination, and the medical institutions that can provide the respective treatments are different. In such cases, the planning function 152 generates a plurality of medical treatment plans by combining the medical institutions and the medical treatment actions. In addition, even for the same medical treatment action, there are cases where a plurality of medical institutions provide the medical treatment action. In such cases, the planning function 152 generates a plurality of medical treatment plans in which the medical institutions are different.

With respect to the medical treatment actions received by the patient, for example, one or a plurality of treatment methods for each of the types of illness and injuries and/or each of symptoms may be registered in advance in the memory 120. In such a case, the planning function 152 searches for a treatment method corresponding to the type of illness and injuries or the symptom of the patient serving as the target of generation of the medical treatment plan from the memory 120, and specifies the medical institution that can provide the treatment method on the basis of the medical institution information. As another example, the planning function 152 may specify one or a plurality of treatment methods corresponding to the type of illness and injuries or the symptom of the patient serving as the target of generation of the medical treatment plan, on the basis of the past medical treatment results in each of the medical institutions in the medical institution information.

As another example, when it is possible for the patient to receive home care without admission to a medical institution or outpatient treatment, the planning function 152 generates a medical treatment plan including home care as a choice.

Each of the medical treatment plans according to the present embodiment is a combination of the medical institutions, medical treatment actions provided to the patient by the medical institutions, and the periods for which the medical institutions provide the medical treatment actions to the patient. A specific example of the medical treatment plans will be described later. The medical treatment plan may be generated based on a result of diagnosis of patient's disease made by a doctor, or the medical treatment plan may be generated by the planning function 152 based on an inspection result that is included in the patient information and registered to the electronic medical chart information with no entry of diagnosis by a doctor.

In addition, the planning function 152 estimates the effect of the medical treatment for the patient for each of the medical treatment plans, on the basis of the medical institution information. For example, the planning function 152 estimates the effects of the medical institutions and the medical treatment actions included in each of the medical treatment plans, on the basis of the information indicating the effects of the past medical treatments in each of the medical institutions. For example, the planning function 152 may estimate that the effect of the medical treatment action is higher as the past recurrence rate or the past mortality rate is lower in the medical institutions and the medical treatment actions. As another example, the planning function 152 may estimate that the effect of the medical treatment action is higher as the number of days of hospitalization is smaller.

In the present embodiment, the planning function 152 rates each of the estimation results of the effects of the medical treatment plans for the patient into the ranks of “best”, “good”, “slightly poor”, “poor”, for example. The method for indicating each of the estimation results of the effects of the medical treatment plans for the patient is not limited thereto. For example, the planning function 152 may use numerical values, such as the number of days of hospitalization, the recurrence rate, and the mortality rate, as the estimation results of the effects for the patient.

In addition, the planning function 152 calculates the total balance of each of the medical treatment plans, on the basis of the medical institution information. For example, the planning function 152 calculates the total balance of the medical treatment plan, by subtracting the sum total of the costs pertaining to the medical treatment actions in the medical institutions in the medical treatment plan from the sum total of the incomes received with the medical institutions in accordance with the medical treatment actions in the medical treatment plan.

As another example, the planning function 152 may calculate the balance of each of the medical institutions included in each of the medical treatment plans. As another example, the planning function 152 may calculate the balance of any specific medical institution for each of the medical treatment plans. For example, the planning function 152 may calculate the balance of the medical institution for which the user works for each of the medical treatment plans, the user being the doctor or the like who has executed the generation process of the medical treatment plans.

The planning function 152 transmits the generated medical treatment plans, and the estimation results of the effects of medical treatments for the patient, the total balance, or the balance of each of the medical institutions for each of the medical treatment plans to the output function 153.

The output function 153 outputs the medical treatment plans generated with the planning function 152 such that the medical treatment plans can be compared. For example, the output function 153 outputs a list of a plurality of medical treatment plans to the terminal device 200 through the NW interface 110 and the network 300, and displays the list of the medical treatment plans in a comparable manner on the display of the terminal device 200.

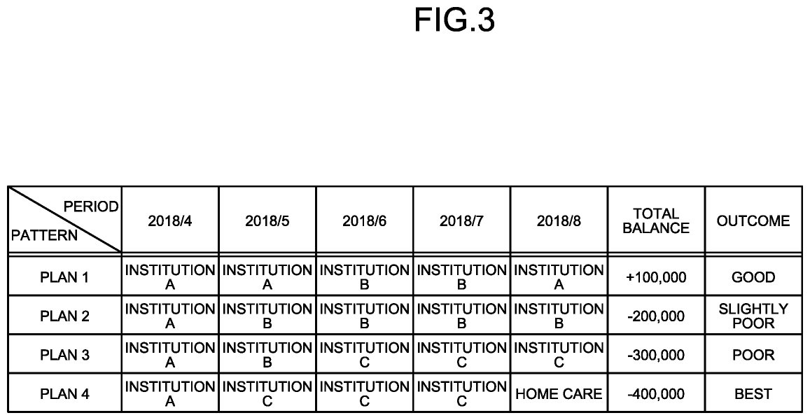

FIG. 3 is a diagram illustrating an example of a list screen of a plurality of medical treatment plans according to the present embodiment. As illustrated in FIG. 3, the output function 153 displays a list of a plurality of medical treatment plans on the screen. For example, suppose that the doctor being the family doctor of the patient and working for the institution A has generated the future medical treatment plans for the patient. As described above, the planning function 152 generates a plurality of medical treatment plans based on combinations of a plurality of medical institutions, medical treatment actions provided to the patient by each of the medical institutions, and the periods for which each of the medical institutions provides the medical treatment actions to the patient.

The time of generation of the medical treatment plans is April 2018, and suppose that the patient goes to the institution A as an outpatient at the point in time. In the example illustrated in FIG. 3, the output function 153 displays the name of the medical institution that provides the medical treatment action to the patient for each month from April 2018 to August 2018. In FIG. 3, the output function 153 displays the institution providing the medical treatment action by the unit of a month as an example, but the time unit for changing the medical treatment action or the medical treatment period is not limited thereto. In addition, the finish time of the treatment period may be different between the medical treatment plans. In addition, in FIG. 3, the output function 153 displays four types of medical treatment plans, but the number of medical treatment plans is not limited thereto.

In addition, the output function 153 displays each of the medical treatment plans on the screen in association with one or more evaluation indexes. The evaluation index for the medical treatment plans according to the present embodiment is an index that allows the user to compare and evaluate the plurality of medical treatment plans. For example, as illustrated in FIG. 3, the output function 153 displays the medical treatment plans each being associated with the total balance of each of the medical treatment plans and the estimation result of the effects of each of the medical treatment plans for the patient. The total balance and the outcome illustrated in FIG. 3 serve as examples of the evaluation indexes.

In the example illustrated in FIG. 3, the output function 153 displays the estimation result of the effects of each of the medical treatment plans for the patient, as the “outcome”. In the example illustrated in FIG. 3, in “Plan 1” to “Plan 4”, the total balance of “Plan 1” is the highest, and the total balance of “Plan 4” is the lowest. In addition, in “Plan 1” to “Plan 4”, the outcome of “Plan 1” is the best, and the outcome of “Plan 4” is the worst. The output function 153 may display only either the total balance or the outcome.

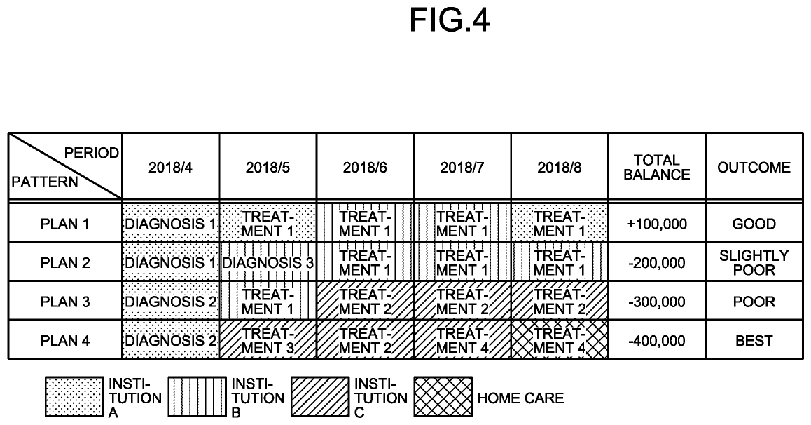

The output function 153 also displays medical treatment actions included in the medical treatment plans. FIG. 4 is a diagram illustrating an example of the list screen displaying medical treatment actions in the medical treatment plans according to the present embodiment. In the example illustrated in FIG. 4, the medical treatment actions are illustrated as “Diagnosis 1”, “Diagnosis 2”, “Diagnosis 3”, “Treatment 1”, “Treatment 2”, “Treatment 3”, and “Treatment 4”, as an example. As illustrated in FIG. 4, different medical institutions may execute the same medical treatment action. For example, when both the “institution A” and “institution B” are capable of providing “Treatment 1” to the patient, the total balance or the outcome may be different depending on which institution, “institution A” or “institution B”, provides “Treatment 1”.

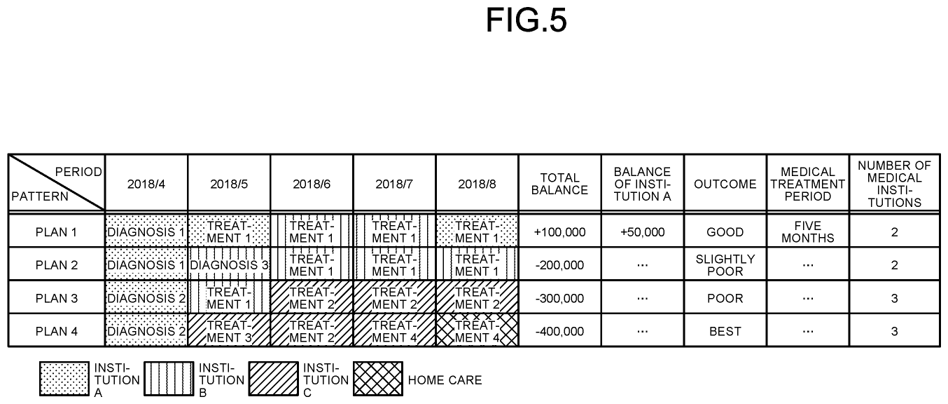

The output function 153 may also display other evaluation indexes for each of the medical treatment plans. FIG. 5 is a diagram illustrating an example of adding items to the list screen of the medical treatment plans according to the first embodiment. In the example illustrated in FIG. 5, the output function 153 additionally displays items “balance of institution A”, “medical treatment period”, and “number of medical institutions”, in comparison with the example illustrated in FIG. 4. Information displayed in these items serves as an example of the evaluation indexes for each of the medical treatment plans, and the output function 153 may further display other evaluation indexes.

For example, the output function 153 switches between the screen displays illustrated in FIG. 3, FIG. 4, and FIG. 5, in accordance with the operator's operation received by the reception function 154 described later.

In addition, the output function 153 increases or decreases the displayed items, in accordance with the operator's operation received with the reception function 154 described later. As another example, the output function 153 switches between the screen displays illustrated in FIG. 3, FIG. 4, and FIG. 5, in accordance with the operator's operation received by the reception function 154 described later.

In addition, the output function 153 sorts the display order of the medical treatment plans on the basis of any one or more of the evaluation indexes, in accordance with the operator's operation received by the reception function 154 described later. The display order may be either the ascending order or the descending order. For example, the output function 153 may display a plurality of medical treatment plans in order according to “the number of medical institutions” included in each of the medical treatment plans, from the smallest. As another example, the output function 153 may sort the display order of the medical treatment plans such that the medical treatment plan with the larger sum of “balance of institution A” is displayed upper in the medical treatment plans having the same “number of medical institutions” in the state in which the medical treatment plans are displayed in order according to “the number of medical institutions” included in each of the medical treatment plans, from the smallest.

The output function 153 may sort the display order of the medical treatment plans on the basis of evaluation indexes that are not displayed, not limited to the evaluation indexes displayed on the display. For example, as illustrated in FIG. 3 and FIG. 4, even when no evaluation indexes other than “total balance” and “outcome” are displayed, the output function 153 may allow sorting of the display order of the medical treatment plans according to the items “balance of institution A”, “medical treatment period”, and/or “the number of medical institutions” that are not displayed, by a method such as displaying a display order setting screen on the display separately.

The output function 153 displays a plurality of medical treatment plans as a list as illustrated in FIG. 3, to enable the operator to easily compare the medical treatment plans. In addition, as described above, the output function 153 displays evaluation indexes for each of the medical treatment plans in the list of the medical treatment plans, to enable easier comparison of the medical treatment plans.

The reception function 154 receives an operation performed by the operator on the terminal device 200 through the network 300 and the NW interface 110. The operator is a doctor or the like, for example. When the operator performs a start operation to start a medical treatment plan generation process from the operation screen displayed on the display of the terminal device 200, for example, the reception function 154 receives the start operation. When the reception function 154 receives the start operation to start the medical treatment plan generation process, the reception function 154 notifies the acquisition function 151 and the planning function 152 of start of the medical treatment plan generation process.

In addition, the reception function 154 receives an operation of changing the display form of the list of the medical treatment plans displayed on the display of the terminal device 200, an operation of adding a display item to the list of the medical treatment plans, and/or an operation of sorting the display order of the medical treatment plans in accordance with one or a plurality of evaluation indexes. The reception function 154 notifies the output function 153 of the received operation.

For example, the acquisition function 151, the planning function 152, the output function 153, and the reception function 154 serving as constituent elements of the processing circuitry 150 are stored in the memory 120, in the form of computer programs executable with a computer. The processing circuitry 150 reads each of the computer programs from the memory 120, and executes the read computer program, to achieve the function corresponding to the computer program. In other words, the processing circuitry 150 in the state of having read each of the computer programs has each of the functions illustrated in the processing circuitry 150 of FIG. 2. FIG. 2 illustrates the case that the single processing circuitry 150 achieves each of the processing functions of the acquisition function 151, the planning function 152, the output function 153, and the reception function 154, but the processing circuitry 150 may be formed of a combination of a plurality of independent processors, and each of the processing functions may be implemented by each of the processors executing each of the computer programs.

The term “processor” used in the explanation described above means circuitry such as a central processing unit (CPU), a graphics processing unit (GPU), an application specific integrated circuit (ASIC), and a programmable logic device (such as a simple programmable logic device (SPLD), a complex programmable logic device (CPLD), and a field programmable gate array (FPGA)). Instead of storing computer programs in the memory 120, the structure may be adopted such that computer programs are directly incorporated into circuitry of the processor. In this case, the processor achieves the functions by reading and executing computer programs incorporated into the circuitry.

The computer programs executed with the processor may be stored and provided in a computer-readable storage medium, such as a compact disc read-only memory (CD-ROM), a flexible disk (FD), a recordable compact disc (CD-R), and a digital versatile disc (DVD), or the like. As another example, the computer programs may be stored in a computer connected to the network, such as the Internet, and may be provided or distributed by download through the network.

The following is an explanation of flow of the process executed in the medical treatment plan support apparatus 100 according to the present embodiment.

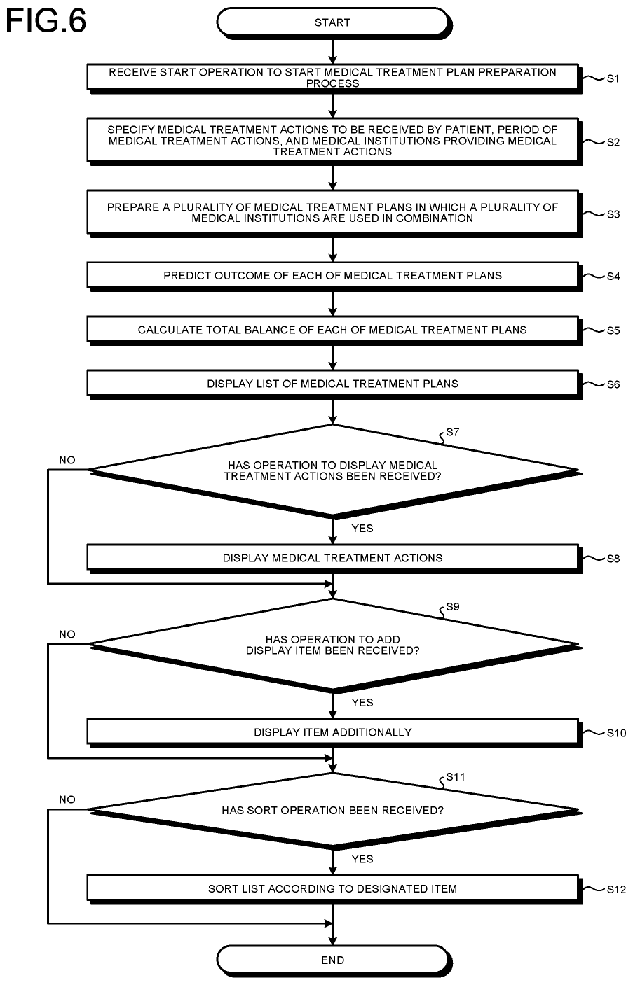

FIG. 6 is a flowchart illustrating an example of flow of a medical treatment plan generation process according to the present embodiment. Before start of the process of the flowchart, the acquisition function 151 acquires the patient information and the medical institution information from the terminal device 200, and stores the pieces of information in the memory 120.

First, the reception function 154 receives a start operation to start medical treatment plan generation process from the operator (S1). At the step, the reception function 154 receives information to specify the patient serving as the target of generation of medical treatment plans. The reception function 154 notifies the planning function 152 of start of the medical treatment plan start process and the information to specify the patient serving as the target of generation of medical treatment plans.

The planning function 152 reads the patient information on the patient serving as the target of generation of medical treatment plans and the medical institution information from the memory 120, and specifies the medical treatment actions to be received by the patient, the periods for which the patient receives the medical treatment actions, and the medical institutions capable of providing the medical treatment actions, on the basis of the patient information and the medical institution information (S2).

Thereafter, the planning function 152 generates a plurality of medical treatment plans in which a plurality of medical institutions are used in combination (S3).

Thereafter, the planning function 152 estimates the outcome of each of the medical treatment plans (S4).

Thereafter, the planning function 152 calculates the total balance of each of the medical treatment plans (S5). The planning function 152 may also calculate the balance of each of the medical institutions included in each of the medical treatment plans. The planning function 152 transmits the generated medical treatment plans, and the outcome and the total balance of each of the medical treatment plans, or the balance of each of the medical institutions included in each of the medical treatment plans to the output function 153.

The output function 153 outputs a list of the medical treatment plans generated with the planning function 152 to the terminal device 200 through the NW interface 110 and the network 300, and displays the list of the medical treatment plans on the display of the terminal device 200 (S6). The output function 153 also displays the total balance and the outcome of each of the medical treatment plans.

Thereafter, the reception function 154 determines whether any operation to display the medical treatment actions has been received (S7).

When the reception function 154 determines that any operation to display the medical treatment actions has been received (“Yes” at S7), the reception function 154 notifies the output function 153 that an operation to display the medical treatment actions has been received. In this case, as illustrated in FIG. 4, the output function 153 displays the medical treatment actions included in the medical treatment plans on the display of the terminal device 200 (S8).

When the reception function 154 determines that no operation to display the medical treatment actions has been received (“No” at S7), the reception function 154 issues no notification to the output function 153.

Thereafter, the reception function 154 determines whether any operation to add a display item has been received (S9). When the reception function 154 determines that any operation to add a display item has been received (“Yes” at S9), the reception function 154 notifies the output function 153 of the display item serving as the target to be added. In this case, the output function 153 additionally displays the display item serving as the target to be added on the display of the terminal device 200, as illustrated in FIG. 5 (S10).

When the reception function 154 determines that no operation to add a display item has been received (“No” at S9), the reception function 154 issues no notification to the output function 153.

Thereafter, the reception function 154 determines whether any operation to sort the display order of the list of the medical treatment plans on the basis of one or a plurality of evaluation indexes has been received (S11). When the reception function 154 determines that a sort operation has been received (“Yes” at S11), the reception function 154 notifies the output function 153 of the evaluation index serving as the basis of sort. In this case, the output function 153 sorts the display order of the list of the medical treatment plans on the basis of the evaluation index serving as the basis of sorting (S12).

When the reception function 154 determines that no sort operation has been received (“No” at S11), the reception function 154 issues no notification to the output function 153. At the step, the process of the flowchart is ended.

In prior art, when a plurality of choices for the treatment method exists or when available medical treatment actions are different between the medical institutions, it is difficult for the family doctor or the like to generate a plurality of medical treatment plans in consideration of these pieces of information.

By contrast, the medical treatment plan support apparatus 100 according to the present embodiment enables support of the doctor or the like to generate medical treatment plans involving a plurality of medical institutions by preparing a plurality of medical treatment plans including medical treatment actions provided by a plurality of medical institutions to the patient, on the basis of the patient information and the medical institution information, and outputting the generated medical treatment plans in a comparable manner.

In addition, the medical treatment plan support apparatus 100 according to the present embodiment displays one or more evaluation indexes in association with each of the medical treatment plans on the display. With this structure, the medical treatment plan support apparatus 100 according to the present embodiment further facilitates the doctor's comparison and consideration of a plurality of medical treatment plans.

In addition, there are cases where the estimated outcome is different according to the details of the medical treatment actions or the medical institutions providing the medical treatment actions to the patient. In such a case, when the family doctor or the like can select the medical treatment plan in consideration of the estimated outcome, more proper medical treatment actions can be provided to the patient.

The medical treatment plan support apparatus 100 according to the present embodiment estimates the effect of the medical treatment for the patient for each of the medical treatment plans on the basis of the medical institution information, and outputs the outcomes of the respective medical treatment plans in association with the medical treatment plans. This structure enables the family doctor or the like to easily compare and consider a plurality of medical treatment plans on the basis of the respective outcomes, on the basis of the results output by the medical treatment plan support apparatus 100. With this structure, the medical treatment plan support apparatus 100 according to the present embodiment enables support of the family doctor or the like to provide more proper medical treatment actions to the patient.

In addition, there are cases where the management of the medical institution in the area serves as a problem. In such a case, for example, there may be needs to avoid the minus total balance of the medical treatment plan.

The medical treatment plan support apparatus 100 according to the present embodiment calculates the total balance of a plurality of medical institutions included in each of the medical treatment plans based on the medical institution information, and outputs the medical treatment plans and the respective total balances of the medical treatment plans in association. With this structure, the medical treatment plan support apparatus 100 according to the present embodiment enables the family doctor or the like to easily recognize whether the total balance is minus for each of the medical treatment plans.

The medical treatment plan support apparatus 100 according to the present embodiment sorts the display order of the medical treatment plans, on the basis of the outcomes, the lengths of the medical treatment periods, the numbers of medical institutions included in the combinations, the total balance of the medical institutions, or the balance of one medical institution included in the medical institutions of the medical treatment plans, in accordance with an operation performed by the operator. The medical treatment plan support apparatus 100 according to the present embodiment enables display of a plurality of medical treatment plans in the display order suitable for the degree of priority of each of the evaluation indexes when the doctor or the like considers the medical treatment plans.

In the present embodiment, the medical institution information is stored in advance in the memory 120, but medical institution information may be acquired from the terminal device 200 when the medical treatment plan support apparatus 100 generates medical treatment plans.

In the present embodiment, the acquisition function 151 acquires the patient information, but the reception function 154 may acquire patient information input to the input screen displayed on the display of the terminal device 200.

In addition, the present embodiment mainly illustrates the case where the family doctor of the patient generates medical treatment plans as an example, but the application example of the medical treatment plan support apparatus 100 according to the present embodiment is not limited thereto. For example, the doctor or the like of an emergency hospital may use the medical treatment plan support apparatus 100 according to the present embodiment to consider transfer of the patient out of the acute phase to another hospital. As another example, the coordinator of the local government or the like may use the medical treatment plan support apparatus 100 according to the present embodiment to consider a plan of the community-based integrated care in which a plurality of medical institutions of the area perform medical treatment on a patient in cooperation with one another.

In addition, in the present embodiment, the planning function 152 of the medical treatment plan support apparatus 100 calculates the total balance of each of the medical treatment plans or the balance of one of the medical institutions, but the planning function 152 may calculate the sum total of a patient's out-of-pocket amounts or the total value of the medical treatment fees in each of the medical treatment plans.

For example, when the output function 153 of the medical treatment plan support apparatus 100 displays the patient's out-of-pocket amount in each of the medical treatment plans, the family doctor or the like is enabled to easily consider the choice with the lower patient's out-of-pocket amount. As another example, when the output function 153 of the medical treatment plan support apparatus 100 displays the sum total of the medical treatment fees in each of the medical treatment plans, the family doctor or the like is enabled to easily consider the choice with the lower sum total of the medical treatment fees. For example, when reduction in medical expenses in the area is considered, the family doctor or the like selects the choice with the lower sum total of the medical treatment fees to contribute to reduction in medical expenses.

In the present embodiment, the display of the terminal device 200 serves as an example of the screen, but the display 140 of the medical treatment plan support apparatus 100 may serve as an example of the screen. In this case, the output function 153 of the medical treatment plan support apparatus 100 may display a plurality of medical treatment plans on the display 140. In addition, the display 140 of the medical treatment plan support apparatus 100 is an example of the hardware display device. Moreover, the display of the terminal device 200 may serve as an example of the hardware display device. In the present embodiment, the family doctor or the like operates the terminal device 200, but he/she may operate the medical treatment plan support apparatus 100.

The community-based integrated care according to the present embodiment includes a medical service offered by an integrated healthcare network (IHN). The IHN is a group formed of, for example, hospitals, doctors, other medical workers, insurance providers, and community organizations. The medical treatment plan support apparatus 100 is used by, for example, a member who joins one IHN. Medical institutions involved in a medical treatment plan may change by addition of a new medical institution or a new insurance provider to the IHN or removal of a medical institution or an insurance provider from the IHN. Moreover, the medical treatment plan support apparatus 100 may display medical treatment plans of each of a plurality of IHNs in a comparable manner.

Moreover, the list screens of the plurality of medical treatment plans as explained with FIGS. 3, 4, and 5 according to the present embodiment may be sharable by a plurality of medical institutions. For example, a list screen of a plurality of medical treatment plans generated by a doctor who works for Institution A may be accessible to a doctor who works for Institution B or C. The medical treatment plan support apparatus 100 enables the same list screen being viewed by a plurality of persons at a plurality of locations, thereby supporting communications occurring, for example, when the family doctor has a consultation with a specialist in remote and when the patient has a consultation with the family doctor via a telephone. It is allowable to restrict persons who are able to access the list screen by log-in user authority, etc. The access restriction can be set for, for example, each user, each list screen, or each medical treatment plan included in the list screen. For example, a user who has higher authority can set a filter on the list screen so that a user who has lower authority cannot see a specific medical treatment plan.

### Second Embodiment

In the present embodiment, the medical treatment plan support apparatus 100 further estimates whether acceptance of the patient is possible or impossible in each of the medical institutions, in accordance with medical resources of each of the medical institutions.

The medical treatment plan support system S according to the present embodiment includes the medical treatment plan support apparatus 100 and a plurality of terminal devices 200a to 200c, in the same manner as the first embodiment. The medical treatment plan support apparatus 100 and the terminal devices 200 according to the present embodiment have the same respective functions as those of the first embodiment.

The medical treatment plan support apparatus 100 according to the present embodiment includes a NW interface 110, a memory 120, an input interface 130, a display 140, and processing circuitry 150, in the same manner as the first embodiment. The processing circuitry 150 includes the acquisition function 151, the planning function 152, the output function 153, and the reception function 154, in the same manner as the first embodiment.

In the present embodiment, medical institution information includes medical resource information regarding medical resources of each of a plurality of medical institutions, in addition to the contents similar to those of the first embodiment. More specifically, the medical resource information includes information as to the number of vacant beds, the reservation state of the medical equipment, human resources of the medical workers, or an inventory quantity of a consumable of each of the medical institutions. The medical equipment is, for example, apparatuses used for radiation treatment, and various types of modality, such as a magnetic resonance imaging (MRI) apparatus and a computed tomography (CT) apparatus. The medical equipment may include medical tools, such as a catheter. The medical workers are, for example, doctors, radiologists, and/or nurses. The consumable is, for example, a mask, protective clothing, an antiseptic solution, or the like. The medical resource information may include not only presence or absence of the medical equipment but also information on newness in version of the medical equipment.

The acquisition function 151, provided with the functions of the first embodiment, acquires medical resource information on each of the medical institutions from the terminal device 200, and stores the information in the memory 120.

The planning function 152 according to the present embodiment, provided with the functions of the first embodiment, estimates whether acceptance of the patient is possible or impossible in each of the medical institutions included in each of the medical treatment plans, on the basis of the medical resource information. The planning function 152 transmits estimation results as to possibleness and impossibleness of acceptance of the patient of each of the medical institutions included in the medical treatment plans to the output function 153. When the state of possibleness and impossibleness of acceptance changes according to the period, the planning function 152 transmits the estimation results as to possibleness and impossibleness of acceptance for the respective periods to the output function 153. In addition, for the medical institution in which acceptance of the patient is impossible, the planning function 152 transmits the reason why acceptance is impossible to the output function 153.

The output function 153 according to the present embodiment, provided with the functions of the first embodiment, outputs information indicating whether acceptance of the patient is possible or impossible in each of the medical institutions. More specifically, the output function 153 displays information indicating whether acceptance of the patient is possible or impossible in each of the medical institutions in association with the list of the medical treatment plans on the display of the terminal device 200.

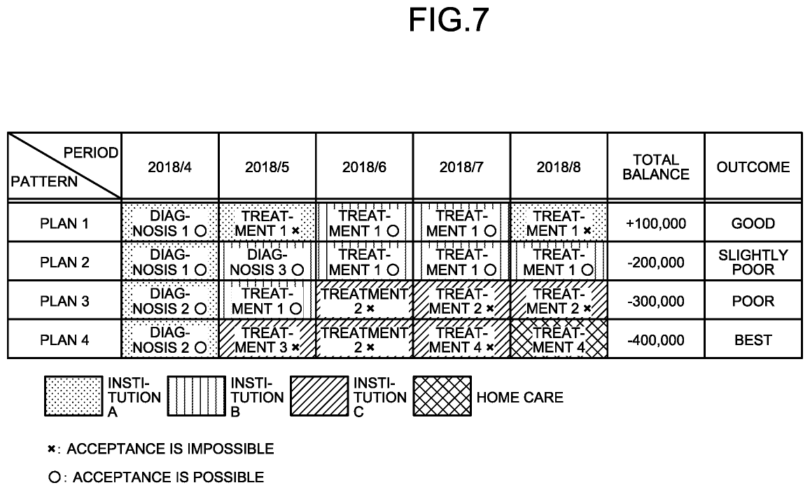

FIG. 7 is a diagram illustrating an example of a list screen of medical treatment plans according to the present embodiment. As illustrated in FIG. 7, the output function 153 displays possibleness and impossibleness of acceptance of the patient in each of the medical institutions in each of the plan periods of the medical treatment plans on the display of the terminal device 200.

In the example illustrated in FIG. 7, the output function 153 displays icons each indicating the period for which the medical institution can accept the patient, and icons each indicating the period for which the medical institution cannot accept the patient. These icons serve as examples of information indicating whether acceptance of the patient is possible or impossible. The display form of the information indicating whether acceptance of the patient is possible or impossible is not limited thereto. The example illustrated in FIG. 7 illustrates the medical treatment actions provided to the patient in the respective plan target periods with the respective medical institutions, but the names of the medical institutions may be displayed as in the example illustrated in FIG. 3.

The reception function 154 according to the present embodiment, provided with the functions of the first embodiment, receives an operator's operation to display the reason why acceptance is impossible, in the medical treatment plans displayed on the display. As an example, the reception function 154 determines that an operation to display the reason why acceptance is impossible has been received, when the reception function 154 receives an operation to select one of the medical treatment plans each involving the medical institution in which acceptance is impossible. As another example, the reception function 154 may determine that an operation to display the reason why acceptance is impossible has been received, when the reception function 154 receives an operator's operation of pressing the icon indicating the period for which acceptance is impossible. When the reception function 154 receives an operation to display the reason why acceptance is impossible, the reception function 154 notifies the output function 153 of reception of an operation to display the reason why acceptance is impossible, and the medical treatment plan serving as the target for which the reason why acceptance is impossible is to be displayed.

The output function 153 outputs the reason why acceptance is impossible in the medical institution, when one of the medical institutions cannot accept the patient. More specifically, when the reception function 154 receives an operation to display the reason why acceptance is impossible, the output function 153 displays the reason why acceptance is impossible for the period for which acceptance is impossible included in the selected medical treatment plan.

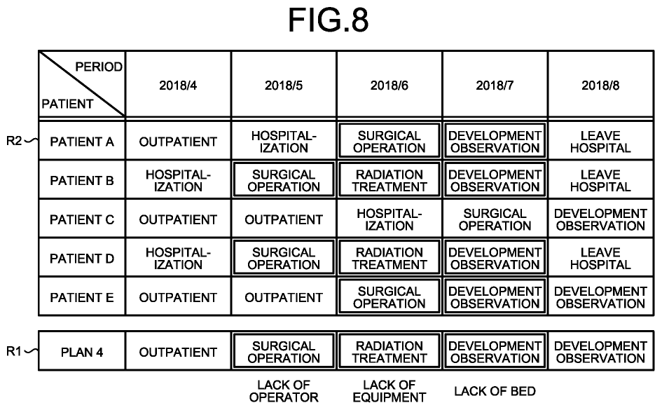

FIG. 8 is a diagram illustrating an example of display of the reason why acceptance is impossible according to the present embodiment. For example, in the example illustrated in FIG. 7, “Plan 4” includes three periods for which acceptance is impossible, that is, “May 2018”, “June 2018”, and “July 2018”. In addition, each of these three periods is a period to which “institution C” is assigned to provide a medical treatment action in “Plan 4.”

In this state, when the operator selects “Plan 4”, the output function 153 displays details of the medical treatment actions of the medical treatment plan “Plan 4” selected by the operator, in a region R1 illustrated in the lower part of FIG. 8.

In addition, the output function 153 displays the reason why acceptance is impossible, for each of the three periods for which acceptance is impossible, that is, “May 2018”, “June 2018”, and “July 2018” included in “Plan 4”. As an example, the reason why acceptance is impossible for “May 2018” is that the “institution C” lacks the operator who is capable of performing the medical treatment action “surgical operation” in the period, that is, the doctor specializing in the surgical operation. The reason why acceptance is impossible for “June 2018” is that the “institution C” lacks the equipment to execute the medical treatment action “radiation treatment”. The reason why acceptance is impossible for “July 2018” is that the “institution C” lacks a bed to hospitalize the patient to perform the medical treatment action “development observation”.

The output function 153 also displays a list of patients for whom reservations have been made in the “institution C” assigned to provide medical treatment actions in “May 2018”, “June 2018”, and “July 2018” being the periods for which acceptance is impossible in the medical treatment plan “Plan 4” selected by the operator, in a region R2 illustrated in the upper part of FIG. 8.

In the operation, the list displayed in the region R2 by the output function 153 is a list of patients using the same medical resources as those of the medical actions to which the “institution C” is assigned to provide them to the patient in “Plan 4”. Specifically, changing the reservation of any of the patient A to the patient E displayed in the region R2 may enable the patient serving as the planning target of “Plan 4” to receive the medical treatment actions in the “institution C”.

The doctor or the like can consider urgency or priority or the like of the patient serving as the planning target of “Plan 4” and the patient A to the patient E displayed in the region R2, and consider changing or cancelling the medical treatment plan. As an example, the doctor or the like can consider urgency or priority or the like of the patient serving as the planning target of “Plan 4” and the patient A to the patient E displayed in the region R2, and consider changing or cancelling the medical treatment plan.

In addition, for some reasons why acceptance is impossible, the doctor or the like can solve the reason why acceptance is impossible by accepting supply of medical resources from other medical institutions, changing the equipment purchase plan, or coordinating beds with other wards.

As another example, when one medical treatment plan includes a period for which acceptance is impossible in a plurality of medical institutions, the output function 153 may display a list of other patients for each of whom reservation has been made, for each of the acceptance-impossible medical institutions included in the selected medical treatment plan.

As another example, the reception function 154 may receive the operator's selection in the unit of one medical institution or one period, even when one medical treatment plan includes a period for which acceptance is impossible in a plurality of medical institutions. In this case, the output function 153 displays a list of other patients for each of whom reservation has been made in the medical institution corresponding to the operator's selection.

As another example, the output function 153 may display only the reason why acceptance is impossible in the region R1, without displaying a list of other patients in the region R2. As another example, the output function 153 may display the reason why acceptance is impossible while maintaining display of the list screen of medical treatment plans illustrated in FIG. 7.

In the explanation described above, the output function 153 displays details of medical treatment actions included in the medical treatment plan when one of the medical treatment plans is selected by the operator, but the output function 153 may display details of medical treatment actions, regardless of presence/absence of selection.

The following is an explanation of flow of a process executed by the medical treatment plan support apparatus 100 according to the present embodiment.

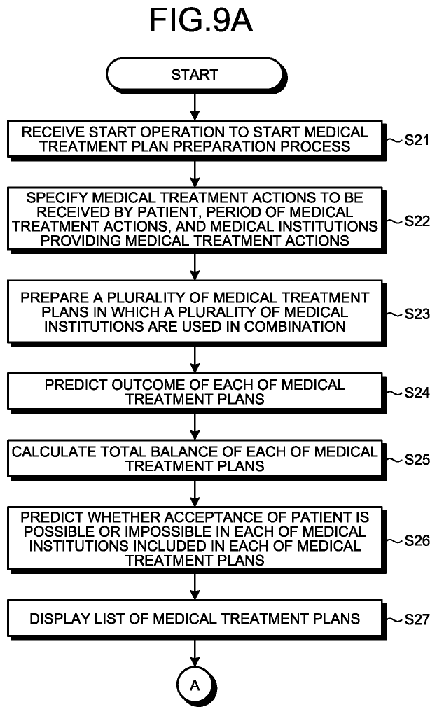

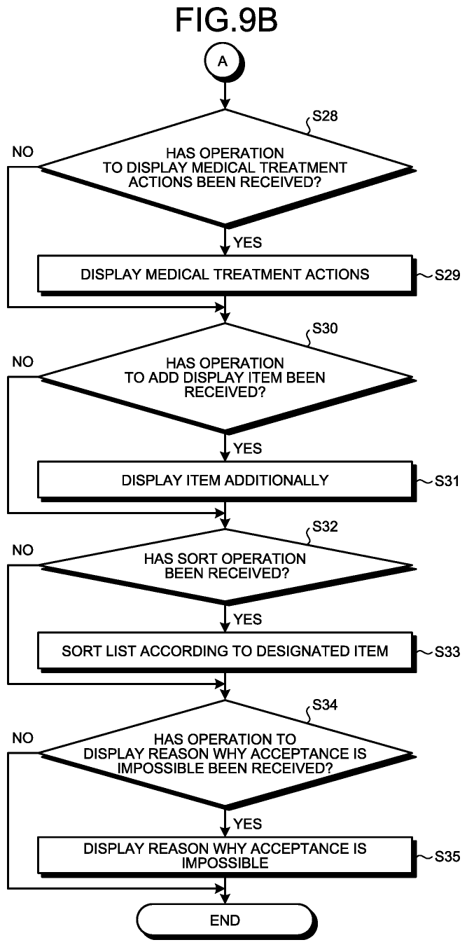

FIGS. 9A and 9B are a flowchart illustrating an example of flow of a medical treatment plan generation process according to the present embodiment. The processing of reception of a start operation of starting medical treatment plan generation processing at S21 to the processing of calculation of the total balance at S25 are the same as the processing at S1 to the processing at S5 explained in FIG. 6 according to the first embodiment.

Thereafter, the planning function 152 estimates whether acceptance of the patient is possible or impossible in each of medical institutions included in each of the medical treatment plans, on the basis of the medical resource information (S26).

Thereafter, the output function 153 outputs a list of medical treatment plans generated by the planning function 152 to the terminal device 200 through the NW interface 110 and the network 300, and displays the list of the medical treatment plans on the display of the terminal device 200 (S27). In addition, in the present embodiment, the output function 153 displays whether acceptance of the patient is possible or impossible in each of the medical institutions in each of the plan periods of the medical treatment plans on the list of the medical treatment plans.

Determination as to presence and absence of reception of the operation to display the medical treatment actions at S28 to sort processing at S33 are the same as the processing at S7 to the processing at S12 explained in FIG. 6 according to the first embodiment.

Thereafter, the reception function 154 determines whether an operation to display the reason why acceptance is impossible has been received (S34).

When the reception function 154 determines that an operation to display the reason why acceptance is impossible has been received (“Yes” at S34), the reception function 154 notifies the output function 153 of reception of the operation to display the reason why acceptance is impossible, and the medical treatment plan serving as the display target.

In this case, the output function 153 displays the reason why acceptance is impossible, for the acceptance-impossible period included in the selected medical treatment plan (S35). In addition, the output function 153 displays a list of patients for each of whom reservation has been made in the medical institution assigned to provide medical treatment actions for the acceptance-impossible period of the medical treatment plan selected by the operator.

When the reception function 154 determines that no operation to display the reason why acceptance is impossible has been received (“No” at S34), the reception function 154 issues no notification to the output function 153. At the step, the process of the flowchart is ended.

As described above, the medical treatment plan support apparatus 100 according to the present embodiment estimates whether acceptance of the patient is possible or impossible in each of the medical institutions included in the medical treatment plans, on the basis of the medical resource information, and outputs possibleness and impossibleness of acceptance of the patient in each of the medical institutions. With this structure, the medical treatment plan support apparatus 100 according to the present embodiment, provided with the effect of the first embodiment, enables support of the doctor or the like to select the medical treatment plan in consideration of the state of possibleness and impossibleness of the patient in each of the medical institution.

In addition, the medical treatment plan support apparatus 100 according to the present embodiment outputs the reason why acceptance is impossible in the medical institution, when acceptance of the patient is impossible in one of the medical institutions. With this structure, the medical treatment plan support apparatus 100 according to the present embodiment enables support of the doctor or the like to consider the future medical resource plan or the like. For example, the doctor or the like is enabled to consider solving the reason why acceptance is impossible, by accepting supply of medical resources from other medical institutions, changing the equipment purchase plan, or coordinating beds with other wards.

Specifically, the medical treatment plan support apparatus 100 according to the present embodiment enables presentation of medical resources required for each of medical institutions, and support of consideration of the facility management plan of each of medical institutions.

### Third Embodiment

In a third embodiment, it is further supported to select a medical treatment plan by a doctor, a care planner, a medical social worker, a staff member of an insurance provider, or the like in accordance with a medical insurance that a patient has.

The medical treatment plan support system S according to the present embodiment includes the medical treatment plan support apparatus 100 and the plurality of terminal devices 200a to 200c, as that of the first embodiment includes. The medical treatment plan support apparatus 100 and the plurality of terminal devices 200 according to the present embodiment have the same functions as those of the first embodiment have.

The medical treatment plan support apparatus 100 of the present embodiment includes the NW interface 110, the memory 120, the input interface 130, the display 140, and the processing circuitry 150, as that of the first embodiment includes.

The memory 120 of the present embodiment further stores insurance information therein in addition to the patient information and the medical institution information. The insurance information at least includes information in which a medical institution covered by insurance is associated with to each medical insurance plan offered by each insurance provider.

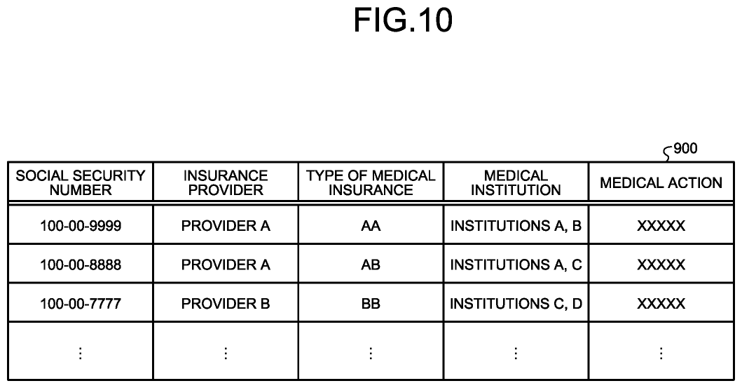

FIG. 10 is a diagram illustrating an example of insurance information 900 according to the present embodiment. As illustrated in FIG. 10, the example of the insurance information 900 includes a social security number (SSN) of a patient, an insurance provider to which the patient subscribes, a plan that is one of medical insurance plans offered by the insurance provider and to which the patient subscribes, a medical institution covered by the medical insurance plan, and a medical action covered by the medical insurance plan, those being associated with each other. The patient's SSN is an example of identification information for identifying the patient. The insurance information 900 may exclude information of a medical action covered by insurance. The insurance information 900 may include information of a medical action uncovered by insurance, instead of a medical action covered by insurance. The medical insurance plan is an example of type of medical insurance in the present embodiment.

The example illustrated in FIG. 10 is merely an example, and the contents of the insurance information 900 are not limited thereto. The insurance information 900 may be, for example, information not including identification information, such as a patient's SSN, and including an insurance provider, a medical insurance plan that is one of medical insurance plans offered by the insurance provider and to which the patient subscribes, a medical institution covered by the medical insurance plan, and details of medical treatment covered by insurance, those being in association with each other. In this case, for example, a doctor, a care planner, a medical social worker, a staff member of an insurance provider, or the like may input an insurance provider to which the patient subscribes and an insurance plan that the patient has to the medical treatment plan support apparatus 100.

The planning function 152 according to the present embodiment determines whether each of the plurality of medical treatment plans is covered by insurance based on the insurance information 900 about the medical insurance that the patient has, in addition to the functions of the first embodiment.

For example, the planning function 152 determines a medical treatment plan is “covered” when all of medical institutes included in the medical treatment plan are indicated by the insurance information 900 to be covered by the insurance plan that the patient has and when all of medical actions included in the medical treatment plan are indicated to be covered by the insurance plan that the patient has.

Moreover, the planning function 152 determines a medical treatment plan is “uncovered” when none of medical institutes included in the medical treatment plan are indicated by the insurance information 900 to be covered by the insurance plan that the patient has or when none of medical actions included in the medical treatment plan are indicated to be covered by the insurance plan that the patient has.

Furthermore, the planning function 152 determines a medical treatment plan is “partially uncovered” when one or more of medical institutes included in the medical treatment plan are uncovered by the insurance plan that the patient has or when one or more of medical actions included in the medical treatment plan is uncovered by the insurance plan that the patient has.

In addition, the planning function 152 calculates a patient's out-of-pocket payment for each of the plurality of medical treatment plans based on the insurance information about medical insurance that the patient has. The planning function 152, for example, subtracts an amount of money for any medical institution and any medical action covered by the insurance plan that the patient has from an amount of money that each of a plurality of medical institutions involved in the medical treatment plan charges the patient for the medical treatment actions, thereby calculating the patient's out-of-pocket payment.

The planning function 152 sends a result of determination on whether each of the medical treatment plans is covered by insurance to the output function 153. In addition, the planning function 152 sends the patient's out-of-pocket payment for each of the plurality of medical treatment plans to the output function 153.

The output function 153 of the present embodiment causes the display 140 to display the plurality of medical treatment plans and whether each of the medical treatment plans is covered by insurance in an associated manner, in addition to the functions of the first embodiment. The output function 153 of the present embodiment further causes the display 140 to display the plurality of medical treatment plans and the patient's out-of-pocket payment for each of the plurality of medical treatment plans in an associated manner. In the present embodiment, the display 140 of the medical treatment plan support apparatus 100 is an example of the screen, but the display of the terminal device 200 may serve as an example of the screen.

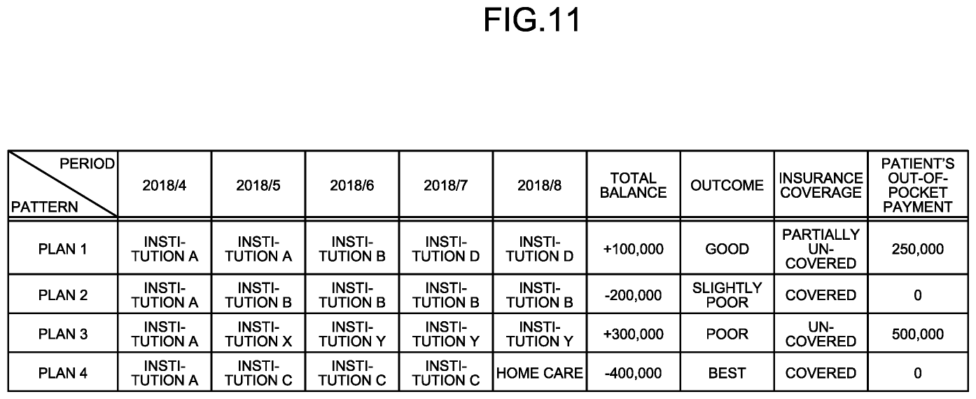

FIG. 11 is a diagram illustrating an example of a list screen of a plurality of medical treatment plans according to the present embodiment. As illustrated in FIG. 11, in the present embodiment, “insurance coverage” and “patient's out-of-pocket payment” are added as evaluation indexes. The “insurance coverage” indicates whether it is covered by insurance, which is determined by the planning function 152. Regarding, for example, “Plan 1” as illustrated in FIG. 1, one or more of the medical institutions or one or more of the medical treatment actions included in the medical treatment plan is uncovered by insurance. In this case, the output function 153 causes the display 140 to display “partially uncovered” for “Plan 1”. Regarding “Plan 2”, all of the medical institutions and all of the medical treatment actions included in the medical treatment plan are covered by insurance. In this case, the output function 153 causes the display 140 to display “covered” for “Plan 2”.

Moreover, the output function 153 may sort or filter the list screen of the plurality of medical treatment plans according to “insurance coverage” or “patient's out-of-pocket payment” and may display the sorted or filtered list screen. The output function 153 may, for example, sort and display the plurality of medical treatment plans according to “patient's out-of-pocket payment” in ascending order.

Furthermore, the output function 153 may filter the list screen of the plurality of medical treatment plans to display any medical treatment plan having the type of “insurance coverage” as “covered” only. In other words, the output function 153 may cause the display 140 to display one or more medical treatment plans covered by the medical insurance that the patient has. The output function 153 causes the display 140 not to display any medical treatment plan of the plurality of medical treatment plans that is uncovered by the medical insurance that the patient has. In this case, it is unnecessary to display the item “insurance coverage” on the screen.

The screen illustrated in FIG. 11 is used, for example, when the medical institution to which the patient goes first is “Institution A” and when a doctor, care planner, or medical social worker who works for Institution A considers a plan of patient's medical treatments in future. The doctor, care planner, or medical social worker who works for Institution A can select from the plurality of medical treatment plans a medical treatment plan covered by the medical insurance that the patient has or a medical treatment plan for which the patient's out-of-pocket payment is low.

A staff member of an insurance provider for whom Institution A or the patient provides information may refer to the scree illustrated in FIG. 11 to generate a medical treatment plan. In this case, by referring to the screen illustrated in FIG. 11, the insurance provider can provide a medical treatment plan covered by medical insurance offered by itself for the patient who has the medical insurance, or the insurance provider can provide information about a medical treatment plan for which the patient's out-of-pocket payment is low for the patient or the doctor of Institution A.

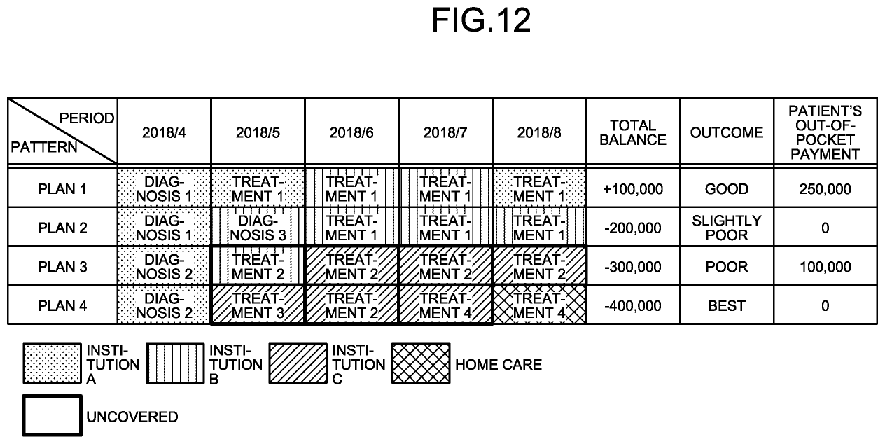

In addition, the output function 153 may display whether medical institutions and medical treatment actions included in medical treatment plans are covered or not by insurance, individually. FIG. 12 is a diagram illustrating another example of the list screen of the plurality of medical treatment plans according to the present embodiment. In the example as illustrated in FIG. 12, when a medical institution or a medical treatment action is uncovered by the insurance plan that the patient has, the output function 153 causes the display 140 to display that it is uncovered by insurance. The output function 153 may display, of the plurality of medical institutions included in the plurality of medical treatment plans, medical institutions covered by insurance and medical institutions uncovered by insurance in a distinguishable manner as such. Moreover, the output function 153 may display, of the plurality of medical treatment actions included in the plurality of medical treatment plans, medical treatment actions covered by insurance and medical treatment actions uncovered by insurance in a distinguishable manner.

As described above, the medical treatment plan support apparatus 100 of the present embodiment determines whether each of a plurality of medical treatment plans is covered by insurance based on the insurance information 900 concerning medical insurance that the patient has. The medical treatment plan support apparatus 100 of the present embodiment causes the display 140 to display the plurality of medical treatment plans and whether each of the plurality of medical treatment plans is covered by insurance in an associated manner. With this configuration, the medical treatment plan support apparatus 100 of the present embodiment allows the user to easily understand whether each of the plurality of medical treatment plans is covered by insurance, in addition to the effects of the first embodiment.

Moreover, the medical treatment plan support apparatus 100 of the present embodiment causes the display 140 to display one more medical treatment plans covered by medical insurance that the patient has, and the medical treatment plan support apparatus 100 causes the display 140 not to display any medical treatment plan uncovered by the medical insurance that the patient has. With this configuration, the medical treatment plan support apparatus 100 of the present embodiment can present to the user a list of medical treatment plans covered by the medical insurance that the patient has only. When the patient does not wish to receive any medical treatment uncovered by the medical insurance, the medical treatment plan support apparatus 100 can improve user's convenience by removing any medical treatment plan uncovered by the medical insurance from the list.

Moreover, the medical treatment plan support apparatus 100 of the present embodiment calculates a patient's out-of-pocket payment for each of a plurality of medical treatment plans based on the insurance information 900 concerning medical insurance that the patient has. The medical treatment plan support apparatus 100 of the present embodiment causes the display 140 to display the plurality of medical treatment plans and the patient's out-of-pocket payment in an associated manner. With this configuration, the medical treatment plan support apparatus 100 of the present embodiment allows the user to easily understand the patient's out-of-pocket payment for each of the plurality of medical treatment plans. For example, even when one or more of medical institutions or one or more of medical treatment actions included in a medical treatment plan is uncovered by the medical insurance that the patient has, there is possibility that the doctor or the patient wishes the medical treatment plan if the patient's out-of-pocket payment is in total within a patient's budget. The medical treatment plan support apparatus 100 of the present embodiment thus can support consideration of the medical treatment plan by the user by displaying the patient's out-of-pocket payment.

The insurance information of the present embodiment is information in which a medical institution covered by insurance is associated with each type of medical insurance offered by each insurance provider. The medical treatment plan support apparatus 100 of the present embodiment causes the display 140 to display, of a plurality of medical institutions included in a plurality of medical treatment plans, medical institutions covered by insurance and medical institutions uncovered by insurance in a distinguishable manner. With this configuration, the medical treatment plan support apparatus 100 of the present embodiment allows the user to understand details of reasoning why the medical treatment plan is uncovered by insurance.

The doctor, the care planner, the medical social worker, and the staff member of the insurance provider are examples of the users of the medical treatment plan support apparatus 100, and the users of the medical treatment plan support apparatus 100 are not limited thereto.

For example, the patient may serve as the user and consider the medical treatment plan using the medical treatment plan support apparatus 100. In this case, the functions of the medical treatment plan support apparatus 100 may be implemented as application software operable by a smartphone. For example, the smartphone may display a list screen of a plurality of medical treatment plans when the patient who goes to a medical institution inputs his/her disease name explained by a doctor and his/her insurance information to the smartphone. It is unnecessary for the smartphone to execute all the functions of the medical treatment plan support apparatus 100. For example, the medical treatment plan support apparatus 100 may include a server and a smartphone that is connected to the server via a network.

At least one of the embodiments described above enables support of the doctor or the like to generate medical treatment plans involving a plurality of medical institutions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical treatment plan support apparatus comprising:
processing circuitry configured to:
generate a plurality of medical treatment plans including medical treatment actions provided by a plurality of medical institutions to a patient, on the basis of patient information indicating a state of the patient and medical institution information regarding the medical institutions; and
cause a hardware display device to display the medical treatment plans in a comparable manner.

2. The medical treatment plan support apparatus according to claim 1, wherein the processing circuitry causes the hardware display device to display each of the medical treatment plans in association with one or more evaluation indexes.

3. The medical treatment plan support apparatus according to claim 2, wherein
the medical institution information is information including past medical treatment results in each of the medical institutions, and information indicating effects of past medical treatments in each of the medical institutions,
the evaluation index is estimation results of effects of medical treatments of each of the medical treatment plans for the patient,
the processing circuitry is configured to
estimate the effects of medical treatments for the patient for each of the medical treatment plans on the basis of the medical institution information, and
cause the hardware display device to display the medical treatment plans and the estimation results of the effects of each of the medical treatment plans for the patient in association.

4. The medical treatment plan support apparatus according to claim 2, wherein
the medical institution information further includes cost pertaining to medical treatment actions in each of the medical institutions, and information on income received with each of the medical institutions in accordance with the medical treatment actions,
the evaluation index is total balance of the medical institutions included in each of the medical treatment plans,
the processing circuitry is configured to
calculate the total balance on the basis of the medical institution information, and
cause the hardware display device to display the medical treatment plans and the total balance of the medical institutions included in each of the medical treatment plans in association.

5. The medical treatment plan support apparatus according to claim 1, wherein the processing circuitry is configured to sort display order of the medical treatment plans on the basis of the estimation results of the effects of medical treatments for the patient, length of a medical treatment period, number of medical institutions included in a combination, the total balance of the medical institutions, or balance of one medical institution included in the medical institutions of each of the medical treatment plans, in accordance with an operator's operation.

6. The medical treatment plan support apparatus according to claim 1, wherein
the medical institution information includes medical resource information regarding medical resources of each of the medical institutions,
the processing circuitry is configured to
estimate whether acceptance of the patient is possible or impossible in each of the medical institutions included in each of the medical treatment plans, on the basis of the medical resource information, and
cause the hardware display device to display information indicating whether acceptance of the patient is possible or impossible in each of the medical institutions.

7. The medical treatment plan support apparatus according to claim 6, wherein, when acceptance of the patient is impossible in one of the medical institutions, the processing circuitry causes the hardware display device to display a reason why acceptance is impossible in the medical institution.

8. The medical treatment plan support apparatus according to claim 1, wherein the processing circuitry is configured to
determine whether each of the medical treatment plans is covered by insurance on the basis of insurance information about medical insurance that the patient has, and
cause the hardware display device to display the medical treatment plans and whether each of the medical treatment plans is covered by insurance in association.

9. The medical treatment plan support apparatus according to claim 1, wherein the processing circuitry is configured to
determine whether each of the medical treatment plans is covered by insurance on the basis of insurance information about medical insurance that the patient has,
cause the hardware display device to display, of the medical treatment plans, one or more medical treatment plans that are covered by the medical insurance that the patient has, and
cause the hardware display device not to display, of the medical treatment plans, any medical treatment plan that is uncovered by the medical insurance that the patient has.

10. The medical treatment plan support apparatus according to claim 1, wherein the processing circuitry is configured to
calculate a patient's out-of-pocket payment for each of the medical treatment plans on the basis of insurance information about medical insurance that the patient has, and
cause the hardware display device to display the medical treatment plans and the patient's out-of-pocket payments in association.

11. The medical treatment plan support apparatus according to claim 8, wherein
the insurance information includes information in which a medical institution covered by insurance is associated with each type of medical insurance offered by each insurance provider, and
the processing circuitry is configured to cause the hardware display device to display, of the medical institutions included in the medical treatment plans, a medical institution that is covered by insurance and a medical institution that is uncovered by insurance in a distinguishable manner.
